(19) United States
(12) Patent Application Publication
(10) Pub. No.: US 2011/0124965 A1
(43) Pub. Date: May 26, 2011

(54) CHEMILUMINESCENCE ENHANCED DETECTION

(76) Inventors: Jason Y. Park, Washington, DC (US); Larry J. Kricka, Devon, PA (US)

(21) Appl. No.: 12/991,568

(22) PCT Filed: May 7, 2009
(86) PCT No.: PCT/US09/43131

§ 371 Date: Dec. 7, 2010

Related U.S. Application Data
(60) Provisional application No. 61/051,395, filed on May 8, 2008.

(51) Int. Cl. A61B 1/06; C12Q 1/42; C12Q 1/34; C12Q 1/37; A61K 49/00

(52) U.S. Cl. 600/160; 435/7.72; 424/9.1

(57) ABSTRACT

Provided are methods for detecting a disease or a condition in a subject, which disease or condition is known to be characterized by abnormal expression of an enzyme The present methods comprise contacting tissue of the subject with a substrate for the enzyme, the substrate chemiluminescing upon reaction with the enzyme, detecting chemiluminescence in the tissue, and, correlating the chemiluminescence to at least one of the presence or absence of the disease or condition in the subject, the stage of the disease or condition in the subject, or the response of the disease or condition in the subject to a therapy regimen The present invention permits sampling, diagnosis, and surveillance of conditions and diseases that are known to be characterized by abnormal expression of at least one enzyme.

### CROSS-REFERENCE TO RELATED APPLICATIONS

This application claims the benefit of priority to U.S. Provisional Application No. 61/051,395, filed May 8, 2008, the entire contents of which are incorporated herein in their entirety.

### FIELD OF THE INVENTION

The present invention pertains to the use of chemiluminescent moieties for the diagnostic visualization and surveillance of tissue of a living subject.

### BACKGROUND OF THE INVENTION

Endoscopy is a standard method for sampling tissue of the upper and lower gastrointestinal tract (esophagus, stomach, colon and rectum) in order to assist the diagnosis of precancerous and cancerous lesions. The method typically involves the examination of luminal structures such as the gastrointestinal tract with a catheter-like device that provides visualization of surface structures. Endoscopy encompasses colonoscopy and esophagogastroduodenoscopy (EGD). The technique may be coupled with devices that allow for ultrasound or confocal microscopic examinations. In addition to visualization, endoscopy can be used for the direction of diagnostic and/or therapeutic procurement of tissue (e.g., tissue biopsy, tissue brushings, cellular aspirations).

An example of endoscopy used in the detection and surveillance of upper gastrointestinal cancer is as follows: a patient presents with symptoms worrisome for esophageal cancer and a physician further investigates by inserting an endoscope into the patient's esophagus and visualizing the luminal surface for changes consistent with or concerning for precancerous or cancerous changes. If changes from a normal appearance are seen, than the physician biopsies the luminal surface that appears different from normal. Biopsies are then submitted for examination of microscopic histology for the evaluation of pathologic change. One weakness of this methodology is that it lacks sensitivity in finding minute foci of diseased tissue while surveying the luminal surface. With respect to this and other aspects of visual examination of gastrointestinal tissue and other tissue types, there remains a great need for technological improvements that allow more precise and timely detection of anomalous tissue development and proliferation.

### SUMMARY OF THE INVENTION

Provided are methods for assessing a disease or a condition in a subject, which disease or condition is known to be characterized by abnormal expression of an enzyme. The present methods comprise contacting tissue of the subject with a substrate for the enzyme, the substrate chemiluminescing upon reaction with the enzyme; detecting chemiluminescence in the tissue; and, correlating the chemiluminescence to at least one of the presence or absence of the disease or condition in the subject, the stage of the disease or condition in the subject, or the response of the disease or condition in the subject to a therapy regimen.

### BRIEF DESCRIPTION OF THE DRAWINGS

FIG. 1 provides data for CSPD/SapphireII chemiluminescence in upper gastrointestinal tract tumors and adjacent tissue.

FIG. 2 shows the results of the measurement of Galacton-Plus/SapphireII chemiluminescence in upper gastrointestinal tract tumors and adjacent tissue.

FIG. 3 illustrates data pertaining to the detection CSPD/SapphireII chemiluminescence in colorectal adenocarcinomas and adjacent tissue

FIG. 4 shows data for Galacton-Plus/SapphireI chemiluminescence in colorectal adenocarcinomas and adjacent tissue

### DETAILED DESCRIPTION OF ILLUSTRATIVE EMBODIMENTS

The present invention may be understood more readily by reference to the following detailed description taken in connection with the accompanying figures and examples, which form a part of this disclosure. It is to be understood that this invention is not limited to the specific products, methods, conditions, or parameters described and/or shown herein, and that the terminology used herein is for the purpose of describing particular embodiments by way of example only and is not intended to be limiting of the claimed invention.

In the present disclosure the singular forms “a,” “an,” and “the” include the plural reference, and reference to a particular numerical value includes at least that particular value, unless the context clearly indicates otherwise. Thus, for example, a reference to “a substrate” or “the substrate” is a reference to one or more of such substrates and equivalents thereof known to those skilled in the art, and so forth. When values are expressed as approximations, by use of the antecedent “about,” it will be understood that the particular value forms another embodiment. As used herein, “about X” (where X is a numerical value) refers to ±10% of the recited value, inclusive. For example, the phrase “about 8” refers to a value of 7.2 to 8.8, inclusive; as another example, the phrase “about 8%” refers to a value of 7.2% to 8.8%, inclusive. Where present, all ranges are inclusive and combinable.

The disclosures of each patent, patent application, and publication cited or described in this document are hereby incorporated herein by reference, in their entirety.

The present invention pertains to the use of chemiluminescent substrates to improve the sampling, diagnosis, and surveillance of conditions and diseases that are known to be characterized by abnormal expression of at least one enzyme. The present invention defines a new category of functional in vivo imaging moieties that are detected optically by luminescence. These moieties include substrates to one or more enzymes that are abnormally expressed as a direct or indirect result of a disease or condition. This novel category of imaging agent is higher in sensitivity and enables a targeted approach to screening for various diseases and conditions, including precancerous and cancerous lesions of the lumen of the esophagus, stomach and intestines. Among other benefits, the present methods therefore enhance the sensitivity of the detection of smaller lesions and permit diagnostic procedures that require fewer invasive measures such as surveillance biopsies.

Chemiluminescence has been used to augment the endoscopic detection of lesions resulting from ischemia or bacterial infection by exploiting light-generating reaction between the chemiluminescent precursor luminol and free radical molecules (Ojetti V, et al. Real Time Endoscopic Imaging of Oxyradical Generation in Pig Stomach During Ischemia-reperfusion. Dig Liver Dis 2003; 35(5):309-13; Zhang Q B, et al. Association of Antral Mucosal Levels of Interleukin 8 and Reactive Oxygen Radicals in Patients Infected with Helicobacter Pylori. Clin Sci (Lond). 1997; 92(1):69-73). In the context of high throughput clinical immunoassay analyzers and commercial Western blotting kits for proteins and Southern and Northern blotting kits for nucleic acids, chemiluminescent assays based on adamantyl 1,2-dioxetane aryl phosphates are one of the principal techniques for detecting and measuring the in vitro activity of alkaline phosphatase, an enzyme that is natively expressed in multiple human tissue sites in normal physiology. Similarly, a 1,2-dioxetane substrate (3-(2′-spiroadamantane)-4-methoxy-4-(3″-beta-D′-galactopyrano-yloxy)phenyl-1,2-dioxetane (AMPGD)) is used for measuring the activity of beta-D galactosidase (lactase and beta-D galactosidase have similar enzymatic activities).

Alkaline phosphatase is an enzyme that is natively expressed in multiple human tissue sites in normal physiology. A recent study involved the use of a fluorescent precipitate to measure normal alkaline phosphatase expression in rat duodenum. See Akiba Y, et al. Duodenal brush border intestinal alkaline phosphatase activity affects bicarbonate secretion in rats. Am J Physiol Gastrointest Liver Physiol 2007; 293 (6):G1223-33. Epub 2007 Oct. 4. The method described by Akiba et al. involved the use of ELF97, a molecule that can be enzymatically cleaved in order to form a fluorescent precipitate that can in turn be excited by an external energy source to produce intense fluorescence. See U.S. Pat. No. 5,316,906; Paragas V B, et al. The ELF-97 Alkaline Phosphatase Substrate Provides a Bright, Photostable, Fluorescent Signal Amplification Method for FISH. J Histochem Cytochem 1997; 45:345-357; Larison K D, et al. Use of a New Fluorogenic Phosphatase Substrate in Immunohistochemical Applications. J Histochem Cytochem 1995; 43:77-83.

However, there have been no studies or findings to the effect that chemiluminescent examination of alkaline phosphatase or other enzymes could be used to evaluate human tissue for the presence of malignancies or other tissue pathologies or conditions. The present invention represents the first and only proposal for the use of chemiluminescent substrates for the detection, evaluation, or monitoring of precancerous or cancerous tissues or of tissues affected by disease states or conditions that are known to be characterized by abnormal enzyme expression.

Numerous studies have shown aberrant expression of small intestine enzymes such as alkaline phosphatase and lactase in precancerous and cancerous tissues of the esophagus, stomach and large intestine (Sugimura T, et al. Intestinal Metaplasia of the Stomach as a Precancerous Stage. IARC Sci Publ 1982; 39:515-30; Kawachi T, et al. Precancerous Changes in the Stomach. Cancer Res 1976; 36(7 PT 2):2673-7; Real F X, et al. Intestinal Brush-border-associated Enzymes: Co-ordinated Expression in Colorectal Cancer. Int J Cancer 1992; 51(2):173-81; Watanabe H, et al. Expression of Placental Alkaline Phosphatase in Gastric and Colorectal Cancers. An Immunohistochemical Study using the Prepared Monoclonal Antibody. Cancer 1990; 66(12):2575-82; Hopwood D, et al. Changes in Enzyme Activity in Normal and Histologically Inflamed Oesophageal Epithelium. Gut 1979; 20(9):769-74). These aberrantly expressed enzymes are enzymatically active. Traditionally, aberrant alkaline phosphatase and lactase expression have been detected at the mRNA and protein level. In addition, studies in which chemiluminescent substrates were used to assess aberrant expression of enzymes have required the purification of the target enzymes from the sample tissue, and indeed it has traditionally believed that purification was necessary. See, e.g., Murray I A, et al. Intestinal Trehalase Activity in a UK Population: Establishing a Normal Range and the Effect of Disease. Br J Nutr 2000; 83:241-245; Karnsakul W, et al. J Ped Gastroenterol Nutr, 2002; 35:551-556; Prasad K K, et al. Brush Border Enzyme Activities in Relation to Histological Lesion in Pediatric Celiac Disease. J Gastroenterol Hepatol 2007 Dec. 5 [Epub ahead of print].

The present invention is directed to methods for assessing a disease or a condition in a subject, which disease or condition is known to be characterized by abnormal expression of an enzyme. The present methods comprise contacting tissue of the subject with a substrate for the enzyme, the substrate chemiluminescing upon reaction with the enzyme; detecting chemiluminescence in the tissue; and, correlating the chemiluminescence to at least one of the presence or absence of the disease or condition in the subject, the stage of the disease or condition in the subject, or the response of the disease or condition in the subject to a therapy regimen.

The assessment of the disease or condition may comprise a correlation of the detected chemiluminescence to the presence or absence of such disease or condition, the stage of development of the disease or condition (which can include the degree or extent of the presence of the disease or condition), the response of the disease or condition in the subject to a therapy regimen, or any other parameter that may be assessed by the detection of chemiluminescence that results from the interaction of a substrate with an enzyme that is abnormally expressed pursuant to the disease or condition. As compared with “normal” expression of the enzyme, “abnormal” expression may refer to higher levels of expression, lower levels of expression, expression that is localized to different physiological locations, a different spatial or temporal pattern of expression, or any other manner of expression that is distinguished from one or more parameters of expression of the enzyme that is observed in the wild type or normal state. For example, numerous studies have determined that certain diseases or conditions can give rise to abnormal localization of enzyme expression.

In normal duodenum, alkaline phosphatase is in apical microvilli (brush border of cells); in metaplasia, alkaline phsophatase expression can be lost from the microvilli. Kumagae Y, et al. Cytochemical Localization of Alkaline Phosphatase in Intestinal Metaplasia of the Human Stomach. Histochem J 1981; 13(1):57-62. In the congenital disease known as sucrase-isomaltase deficiency; there is a mutation resulting in an amino acid substitution. This amino acid substitution results in an abnormal protein folding pattern which leads to increased turnover and abnormal intracellular sorting. Rather than be placed in the normal apical position in the cell's brush border, the enzyme is placed basolaterally. This basolateral location precludes access of the enzyme to the lumen of the intestine. Keiser M, et al. Altered Folding, Turnover, and Polarized Sorting Act in Concert to Define a Novel Pathomechanism of Congenital Sucrase-Isomaltase Deficiency. J Biol Chem 2006; 281 (20):14393-9. In another study, cellular localization of sucrase-isomaltase was performed by immunohistochemistry in esophageal intestinal metaplasia and adenocarcinoma. It was determined that in intestinal metaplasia, sucrase-isomaltase is localized to the apical membrane, while in esophageal adenocarcinoma, sucrase-isomaltase is localized diffusely within the cytoplasm. Wu G D, et al. Sucrase-isomaltase Gene Expression in Barrett's Esophagus and Adenocarcinoma. Gastroenterology 1993; 105(3):837-44. It has also been determined that aminopeptidase N has different cellular localization in non-cancerous colonocytes compared to neoplastic and pre-neoplastic intestines. It was found that aminopeptidase N was localized to the basal membrane of colonocytes of intestines not involved by tumor. In contrast, in colonocytes distal to tumors, aminopeptidase N was localized to the surface epithelium. Furthermore, within cells of preneoplastic adenomas or adenocarcinomas, aminopeptidase N was localized to the surface epithelium. Quaroni A, et al. Expression and Different Polarity of Aminopeptidase N in Normal Human Colonic Mucosa and Colonic Tumors. Int J Cancer 1992; 51(3):404-11.

In some embodiments of the present invention, the substrate may be administered to the subject intravascularly (e.g., intravenously) so that the substrate will contact a basolateral surface of the cells of the tissue and thereby react with any enzyme that is basolaterally expressed. In other embodiments, the substrate may also or alternatively be administered to the subject intraluminally (e.g., perorally) so that the substrate will contact an apical surface of the cells of the tissue and thereby react with any enzyme that is apically expressed. In addition, substrate that is presented intraluminally and/or basolaterally may be transported to the various cytosolic locations (e.g., endoplasmic reticulum, golgi apparatus, etc.) before enzymatic activation. In accordance with the present invention, the method of administration of the substrate may therefore be selected to effect these and other objectives.

The present methods may involve the use of a substrate for any enzyme that is aberrantly or abnormally expressed as a direct or indirect result of a disease or condition. Nonlimiting examples of suitable enzymes include alkaline phosphatase, beta-galactosidase, glucosidase, glucoronidase, aryl sulfatase, trehalase, maltase, dipeptidyl peptidase-IV, aminopeptidase N, endopeptidase F, and sucrase-isomaltase. As discussed supra, numerous in vitro studies have shown aberrant expression of small intestine enzymes such as alkaline phosphatase and lactase in precancerous and cancerous tissues of the esophagus, stomach and large intestine. Other non-chemiluminescent (e.g., colorimetric) studies have demonstrated that other enzymes, including glucosidase, glucuronidase, and aryl sulfatase are expressed in precancerous and cancerous tissues (De Martinez N R, et al. Alterations of Glycosidases in Benign, Premalignant and Malignant Human Lesions. Cancer Detect Prey 1984; 7 (1):37-43; Schwartz M K. Enzymes in Colon Cancer. Cancer 1975; 36: 2334-6; Borkje B, et al. Enzyme Activities in Biopsy Specimens from Large-bowel Mucosa in Colorectal Adenomas and Carcinomas. Scand J Gastroenterol 1987; 22(5):533-8; Finch P J, et al. Gastric Enzymes as a Screening Test for Gastric Cancer. Gut 1987; 28(3):319-22). See also Watanabe H. Experimentally Induced Intestinal Metaplasia in Wistar Rats by X-ray Irradiation. Gastroenterology 1978; 75(5): 796-9 (intestinal metaplasia in the stomach caused by radiation resulted in expression of lactase, trehalase, maltase and sucrase); Kawachi T, et al. Precancerous Changes in the Stomach. Cancer Res 1976; 36(7 PT 2):2673-7 (intestinal metaplasia associated with gastric cancer has expression of sucrase, maltase, trehalase, and alkaline phosphatase); Real F X et al. Intestinal Brush-border-associated Enzymes: Co-ordinated Expression in Colorectal Cancer. Int J. Cancer. 1992; 51 (2):173-81 (the enzymes dipeptidyl peptidase-IV, aminopeptidase N, endopeptidase F, sucrase-isomaltase and alkaline phosphatase are frequently expressed in colorectal cancers).

Chemiluminescent substrates have been previously characterized for many of these enzymes (Olesen C E, et al. Detection of Beta-galactosidase and Beta-glucuronidase Using Chemiluminescent Reporter Gene Assays. Methods Mol. Biol. 1997; 63:61-70; Kricka L J, et al. Chemiluminescent Assay of Enzymes Using Proenhancers and Pro-anti-enhancers. J Biolumin Chemilumin 1991; 6(4):231-8; De Martinez N R, et al., Alterations of Glycosidases in Benign, Premalignant and Malignant Human Lesions. Cancer Detect Prey 1984; 7 (1):37-43; Kricka L J, Voyta, J C, Bronstein, I. Chemiluminescent Methods for Detecting and Quantitating Enzyme Activity. Methods Enzymol 2000; 305:370-390; Bronstein, I, Fortin, J, Voyta, J C, Juo, RR, Edwards, B, Olesen, CEM, Lijam, N, Kricka, L J Chemiluminescent Reporter Gene Assays: Sensitive Detection of the Gus and Seap Gene Products. Bio Techniques 1994; 17:172-177; Bronstein I, Fortin J, Voyta J, Olesen C, Kricka L J Chemiluminescent Reporter Gene Assays for Beta-galactosidase, Beta-glucuronidase and Secreted Alkaline Phosphatase. J Biolumin Chemilumin 1994; 9:291; Bronstein I B, Fortin J, Stanley P E, Stewart G S A B., Kricka L J Chemiluminescent and Bioluminescent Reporter Gene Assays. Anal Biochem 1994; 219:169-181). For example, alkaline phosphatase cleaves the phosphate group of the adamantyl 1,2-dioxetane aryl phosphate substrate to produce an unstable phenoxide. This phenoxide decomposes via scission of the 1,2-dioxetane ring to produce adamantanone and a fluorescent phenoxy ester in an electronically excited state. The latter intermediate decays to the electronic ground state and the excess energy is emitted as a glow of light (Bronstein I, et al. 1,2-Dioxetanes: Novel Chemiluminescent Enzyme Substrates. Applications to Immunoassays. J Biolumin Chemilumin. 1989; 4(1):99-111). This emission of light can be further improved by enhancer molecules such as polymers (e.g., poly[vinylbenzyl(benzyldimethyl ammonium) chloride]) that stabilize the light signal. The sensitivity of in vitro alkaline phosphatase chemiluminescent assays is greater than picomolar (10⁻¹⁵).

However, there have been no studies or findings to the effect that chemiluminescent examination of such enzymes could be used to evaluate human tissue in vivo for the presence of malignancies or other tissue pathologies or conditions. The aberrant expression of numerous other enzymes are known to be associated with particular disease states or conditions, and any such enzyme, once identified as one whose expression parameters may be correlated to a particular disease or condition, may be a subject of study pursuant to the instantly disclosed methods.

Any chemiluminescent substrate for such suitable enzyme may be utilized, and in numerous instances chemiluminescent substrates for particular enzymes have been characterized for use in conjunction with in vitro assays in which purified enzyme is studied. As discussed supra, 1,2-dioxetane substrates, including adamantyl 1,2-dioxetane substrates and adamantyl 1,2-dioxetane aryl phosphate substrates have been identified as chemiluminescent substrates for such enzymes as alkaline phosphatase and lactase/beta-D galactosidase. Various other chemiluminescent moieties have been identified as substrates for particular enzymes, including enzymes that are associated with particular disease states or conditions, and one skilled in the art will readily appreciate that once an enzyme has been identified, a suitable chemiluminescent substrate may be selected or prepared as a matter of course or routine experimentation.

The diseases or conditions that may be detected in accordance with the present methods are those that are characterized by abnormal expression of an enzyme. For example, various conditions involving uncontrolled or aberrant cell growth or proliferation, i.e., neoplasia, are characterized by abnormal expression of one or more enzymes. Thus, precancerous or cancerous conditions may be detected in accordance with the present invention. A chemiluminescent in vivo assay of enzyme activity in accordance with the present invention may be used to detect many other diseases or conditions than cancer or precancer. For example, the decrease or absence of intestinal enzymes has been well established in gastrointestinal disease. Nonlimiting examples of diseases or conditions marked by changes in enzyme levels that may be detected pursuant to the present methods include: primary enzyme deficiencies; autoimmune diseases (psoriatic enteropathy; celiac sprue); inflammatory diseases (ulcerative colitis, Crohn's disease); infectious diseases (Whipple's disease, giardiasis, viral infections, amoebiasis); pernicious anemia; protracted diarrhea; and, malnutrition in infancy (see, e.g., Keane R, et al. Intestinal Lactase, Sucrase and Alkaline Phosphatase in Relation to Age, Sex and Site of Intestinal Biopsy in 477 Irish Subjects. J Clin Pathol 1983; 36:74-77; O'Grady J G, et al., Intestinal Lactase, Sucrase, and Alkaline Phosphatase in 373 patients with Coeliac Disease. J Clin Pathol 1984; 37:298-301; Miki K, et al. Alkaline Phosphatase Isoenzymes in Intestinal Metaplasia and Carcinoma of the Stomach. Cancer Res 1976; 36: 4266-4268). Any disease or condition that is characterized by abnormal expression of an enzyme may be detected in accordance with the present methods.

The tissue with which the substrate is contacted may be selected from any body tissue to which a substrate can be directly or indirectly delivered. As the substrate may be delivered locally or systemically, any body tissue may be assessed in accordance with the present invention. In some embodiments, the tissue may be adjacent to a fluid- or air-filled body cavity that is accessible from the outside of the body, e.g., transvascularly, transluminally, percutaneously, transdermally, orally, nasally, rectally, or parenterally, by optical imaging equipment. For example, the present methods may be used in connection with the selection of biopsy sites of the esophagus when screening for intestinal metaplasia (i.e., precancerous or cancerous) lesions. Alternatively, in the context of screening for recurrent esophageal or gastroesophageal junction disease (e.g., status-post endomucosal resection, photodynamic therapy, surgical resection), the present methods may be used for high-sensitivity detection of areas of interest, such as diseased tissue. The present methods may be used to detect a disease or condition by detection of chemiluminescence in many other specific tissues, including tissues found in the esophagus, gastric system, colorectal system, intestines, sinonasal cavity, oral cavity, oropharynx, respiratory tract, hepatobiliary system, pancreatic ducts, urethra, bladder, ureters, renal pelvis, vagina, cervix, uterus, peritoneal cavity, pleural cavity, or any combination thereof.

Contacting the tissue with the substrate may be accomplished by any means that achieves physical contact there between. A dosage quantity of substrate may be administered to a subject one or more times in order to bring about contact between the substrate and the tissue. For example, depending on the desired regimen, a fixed quantity of substrate may be administered to a subject one or more times, or varying quantities of substrate may be administered over time in order to achieve the desired result with respect to contacting the tissue with the substrate. The quantity of substrate per dose and the total quantity of substrate, respectively, to be administered to the subject will vary depending on such factors as the location of the target tissue(s), the physical characteristics of the subject, the type of substrate, the identity of the enzyme, the dosage form, and the delivery method, and those skilled in the art will readily appreciate how to determine the appropriate administration parameters with respect to the substrate. The substrate may be administered by an individual other than the subject, such as by a physician, clinician, or nurse, or may be self-administered by the subject.

The substrate may be administered by any conventional means available for use in conjunction with pharmaceuticals, either as individual therapeutic agents or in a combination of therapeutic agents. For example, a substrate may be administered as the sole active agent in a pharmaceutical composition, or can be used in combination with other therapeutically active ingredients, where medically appropriate. The substrate may be administered to the subject in a pure or isolated form, or may be administered with other pharmaceutical agents, such as carriers, diluents, or excipients. A carrier, excipient, or diluent can include suitable pharmaceutical additives such as solubilizers, emulsifiers, buffers, preservatives, sweeteners, flavoring agents, suspending agents, thickening agents, colors, viscosity regulators, stabilizers, or osmo-regulators. The intended mode of delivery of the substrate and the identity and location of the tissue with which the substrate is contacted may make some formulations comprising a substrate particularly desirable. For example, formulations of a substrate and a buffered viscous medium may be used in order to facilitate the formation of a coating on a surface of the gastrointestinal tract. The formation of a coating increases the duration of the period of contact between the formulation comprising the substrate and the tissue, which in turn increases the likelihood of reaction between the substrate and any existing enzyme and the generation of a chemiluminescent signal. The selection of the dosage form by which the substrate is administered to the subject may depend on various factors that will be readily appreciated by those skilled in the art.

In addition, substrate may be delivered by use of micelles or liposomal complexes. For example, substrate molecule may be loaded into micelles or liposomes that in turn may be administered to the subject. Depending on the characteristics of the substrate molecule, the substrate may also or alternatively be adhered to the outer surface of a micelle or liposome or embedded within the phospholipid bilayer.

An additional mode of delivery of substrate includes associating substrate with a carrier structure, such as by attaching substrate molecule to the surface or within surface features of micro-, meso-, and/or nanostructures, and administering such structures to the subject. Appropriately-sized structures of such types can be constructed from a wide variety of materials, including, for example, carbon, silica, peptides, metals (e.g., palladium gold, lead zirconate titanate, and barium titanate), and organic polymers. Methods of constructing such structures are known in the art, for example, by pyrolytic or membrane deposition methods, by template synthesis, by wetting of porous templates or in-pore polymerization, by electroless deposition, or by sol-gel chemistry (Martin C R. Nanomaterials—A Membrane-Based Synthetic Approach. Science 1994; 266:1961-1966; Hulten J C, et al. A General Template-based Method for the Preparation of Nanomaterials. J Mater Chem 1997; 7:1075-1087; Cepak V, et al. Fabrication and Characterization of Concentric-tubular Composite Micro and Nanostructures Using the Template-synthesis Method. J Mater Res 1998; 13: 3070-3080; Nicewarner-Pena et al. Submicrometer Metallic Barcodes. Science 2001; 294: 137-141; Mitchell D T, et al. Smart Nanotubes for Bioseparations and Biocatalysis. J Am Chem Soc 2002; 124:11864-5) and are thus not described herein in detail. Numerous types of nanostructures are known in the art and may include Buckminsterfullerenes, nanowires, nanorods, nanotubes, branched nanowires, nanotetrapods, nanotripods, nanohorns, nanobipods, nanocrystals, nanodots, quantum dots, nanoparticles, nanoribbons, or any combination thereof, and may comprise carbon or silicon or both. Additionally or alternatively, such structures as described above may be used to enhance the luminescent signal generated by the substrate upon reaction with an enzyme. This technique is described with respect to nanostructures in PCT Pub. No. WO 2006/116683, which is incorporated herein by reference in its entirety, and which may be adapted to micro- or mesostructures as well. A similar example may be found in U.S. Pub. No. 2006/0216768 (U.S. Ser. No. 11/221,895, filed Sep. 9, 2005) (incorporated by reference herein in its entirety), which discloses the attachment of chemiluminescent substrates to the surface of fluorescent quantum dots; upon enzymatic cleavage, the substrate gives off energy that energizes the fluorescent quantum dot and results in the emission of a lower energy wavelength.

The instant methods may also comprise contacting the tissue with an enhancer molecule that stabilizes and/or augments the luminescent signal produced by the substrate upon reaction with its respective enzyme(s). The enhancer molecule may be administered contemporaneously with the substrate, or the two moieties may be administered separately. Numerous organic and polymer enhancers of chemiluminescence are known among those skilled in the art and may be selected as dictated by the circumstances of the chosen method, e.g., depending on the identity of the substrate. The enhancer will be present in an amount which enhances light production from the substrate in the presence of the enzyme and/or decreases background chemiluminescence. It is highly preferred that the enhancer molecule is pharmaceutically acceptable for administration to human beings. One example of a polymer enhancer is poly[vinylbenzyl(benzyldimethyl ammonium)chloride]. Other enhancers include, for example, phenolic compounds such as those disclosed in U.S. Pat. No. 5,306,621, incorporated herein by reference in its entirety, as well as boronic compounds, such as those disclosed in U.S. Pat. No. 5,629,168, incorporated herein by reference in its entirety. Other enhancers include 6-hydroxybenzothiazole, substituted phenols, such as those disclosed in U.S. Pat. No. 4,598,044, incorporated herein by reference in its entirety; aromatic amines including those disclosed in U.S. Pat. No. 4,729,950, incorporated herein by reference in its entirety; and phenols substituted in ortho and/or para positions by imidazolyl or benzimidazolyl (U.S. Pat. No. 5,043,266, incorporated herein by reference in its entirety).

Administration of a substrate may be local or systemic. Exemplary, nonlimiting modes of administration include intravenous, intramuscular, subcutaneous, intraocular, intrasynovial, transepithelial including transdermal, ophthalmic, sublingual, buccal, topically including ophthalmic, dermal, ocular, rectal (e.g., as a suppository or enema), nasal, via inhalation, or rectal systemic.

Depending on the mode of administration and dosage form of the substrate, it may be desirable for the subject to undergo certain preparations prior to administration. For example, if the substrate is to be contacted with any tissue associated with the gastrointestinal tract, the subject may undergo certain preparations to clear the gastrointestinal tract, such as abstinence from eating, bowel preparations, ingestion of mucolytic agents, and the like, as will be readily appreciated by those skilled in the art.

In some instances, it may be desirable to selectively inhibit certain subtypes of the enzyme by contacting the tissue with an inhibitor, so that only certain enzyme subtypes are able to react with the substrate. For example, multiple types of alkaline phsophatase have been identified; some are tissue-specific, such as intestinal alkaline phosphatase, while others are tissue non-specific. See Moss D W. Perspectives in Alkaline Phosphatase Research. Clin Chem 1992; 38:2486-2492. One type of tissue non-specific alkaline phosphatase is expressed in neutrophils (leukocytes). The presence of numerous neutrophils at the site of the proposed in vivo analysis can obscure the detection of ectopic expression of intestinal alkaline phosphatase. Accordingly, an inhibitor may be used to neutralize the activity of neutrophil-expressed alkaline phosphatase vis-à-vis the substrate; numerous inhibitors have been described that can be used to selectively inhibit tissue non-specific alkaline phosphatase to allow for the detection of intestinal alkaline phosphatase. See Goldstein D J, et al., Expression of Alkaline Phosphatase Loci in Mammalian Tissues. PNAS 1980; 77:2857-2860; Doellgast G J and Meis P J. Use of specific Inhibitors to Discriminate Alkaline Phosphatase Isoenzymes Originating from Human Liver, Placenta and Intestine: Absence of Meconial Alkaline Phosphatase in Maternal Serum. Clin Chem 1979; 25: 1230-1233. Those skilled in the art will readily appreciate that inhibitors of other enzymes may be used under conditions where it is desirable to inhibit the activity of one or more subtypes of such enzymes.

The present methods may further comprise contacting the tissue with a contrast agent. Contrast agents may be used to augment the visual effect of the chemiluminescence that is produced by the substrate upon reaction with the enzyme. Contrast agents may include radio-opaque moieties such as barium sulfate. A contrast agent may be administered contemporaneously with the substrate or may be separately administered. The use of a contrast agent can enable the acquisition of images produced using x-ray or computed tomography methodologies; such images can be considered in tandem with the visual results of a chemiluminescent study. For example, electronic or film-derived images produced using x-ray or CT imaging techniques as enabled through the use of a contrast agent may be overlayed with electronic or film-derived images resulting from the detection of chemiluminescence produced by the substrate/enzyme reaction; the resulting overlay provides the clinician with two parameters for evaluation of the subject tissue.

As used throughout the present disclosure, unless otherwise specified, “substrate” is intended to embrace one or more of such substrates and/or equivalents thereof. Accordingly, the present methods may comprise contacting the tissue with a substrate for the enzyme, and contacting the tissue with one or more additional substrates for the enzyme. In another embodiment, the present methods may comprise contacting the tissue with a substrate for the enzyme, and contacting the tissue with one or more substrates for a second enzyme. The additional/second substrate(s) may be contacted with the tissue contemporaneously with the first substrate, or may be contacted with the tissue before or after the first substrate has been contacted with the tissue. If the first substrate is administered in one or more episodes over time, one or more of these episodes may also comprise the administration of the additional/second substrate(s), or the additional substrates may be administered in one or more episodes that are distinct from each of the episodes involving the administration of the first substrate. The additional substrate(s) may represent one or more different classes of substrate than the first substrate; one class of substrate may vary from a different class of substrate in a number of ways, including the molecular mechanism by which the enzyme and substrate react, the mechanism by which the substrate generates chemiluminescence upon reaction with the substrate, the characteristics of the chemiluminescence produced by the substrate, the general medical compatibility of the substrate with patients or the medical compatibility of the substrate with a particular class of patient, and the like, as will be recognized among those skilled in the art. The second substrate may be a substrate for an enzyme that is abnormally expressed as a direct or indirect result of the disease or condition, or may be a substrate for an enzyme that is abnormally expressed as a direct or indirect result of a second disease or condition.

The chemiluminescence that is generated as a result of the reaction of the enzyme with the substrate may be detected by any mechanism that can detect photons. A standard method of visually sampling tissues, for example in the upper and lower gastrointestinal tract (the esophagus, stomach, colon, and rectum) is endoscopy. Endoscopes of various types may be used to detect chemiluminescence emitted from a substrate that has been contacted with any of a number of different tissue types at various physiological locations. Those skilled in the art will readily appreciate the characteristics and uses for various different types of endoscopes. Endoscopes for use in connection with the present methods may be configured for effecting confocal microscopy, ultrasound, delivery of a substance to the tissue (e.g., contacting a substrate, enhancer, contrast agent, or any other desired substance with the tissue by spraying or otherwise emitting the substance), retrieval of at least a portion of the substrate after administration, procurement of tissue (e.g., tissue biopsy, tissue brushing, cellular aspiration). For example, when the endoscope is configured for effecting confocal microscopy, normal endoscopic detection of chemiluminescence can be coupled with or succeeded by microscopic examination of discrete areas of interest on, in, or near the tissue, including individual cells or cell groupings; in this way, the phenotype of relatively small portions of a tissue may be determined. Other devices and techniques may be used to detect the chemiluminescence resulting from the reaction of the substrate with the enzyme. Nonlimiting examples include free-floating capsule/pill endoscopes that can be delivered in an active form or configured for time-delayed activation (see, e.g., U.S. Pat. Nos. 4,278,077; 5,179,938; 5,604,531; 6,939,292; 7,153,259; and, 7,160,258), tomography, and generic detection devices such as photomultiplier tubes. The present invention is not limited to any particular means of detecting the chemiluminescence in the tissue.

The process of detecting photons generated by the chemiluminescence of the substrate may include detection of light at visible wavelengths, and may also or alternatively include detection of light at nonvisible wavelengths, such as ultraviolet or infrared. The detection may occur at a wavelength or range of wavelengths that corresponds to the wavelength of light emitted by the chemiluminescent substrate. The detection of chemiluminescence need not occur within the subject; if the chemiluminescence occurs at a wavelength that has a high rate of penetration of tissue, an externally situated device may be used, e.g., at the surface of the subject's skin to detect the chemiluminescence. The chemiluminescence detection mechanism may include a filter for the wavelength of light that corresponds to the chemiluminescence emitted by the substrate; for example, an endoscope may include a 470 nm filter, which corresponds to the emission wavelength of a particular 1,2-dioxetane substrate. Pursuant to this approach, endoscopic light emission for imaging of the tissue at visible wavelengths can occur at, for example, 550 to 700 nm, such that the endoscopic light emission is readily distinguishable from the chemiluminescent light.

The chemiluminescence that is generated by the substrate may itself have therapeutic effects. For example, chemiluminescence at the surface of a tissue, especially in the upper gastrointestinal tract, can cause the death of bacteria at the tissue surface. Some enzymes are not expressed in a particular tissue except where a disease or condition is present, and the disease or condition itself may be at least partially caused by the presence of pathogenic organisms; in such instances, chemiluminescent light will be emitted at tissues where the enzyme is expressed, and therefore where the disease or condition is present and at least partially attributable to the presence of pathogen. Chemiluminescent irradiation of pathogenic organisms may therefore be achieved in the self-selecting manner just described.

Subsequent to the detection of chemiluminescence in vivo, it may be desirable to subject the tissue to a washing procedure, retrieve the wash solution, and subject the retrieved wash solution to in vitro analysis. Alternatively, if the substrate was delivered in liquid form or as part of a fluid composition, the substrate-containing liquid can be retrieved and subjected to in vitro analysis. In vitro analysis of the retrieved wash or substrate-containing liquid may be used to determine whether additional quantities of reacted (chemiluminescent) or unreacted substrate may be present and thereby effect a comprehensive assessment of enzyme expression.

The correlation of the chemiluminescence that is detected in the tissue to the absence or presence of the disease or condition can comprise applying what is known about expression levels, expression localization characteristics, and/or temporal or physical expression patterns of the enzyme as a direct or indirect result of the presence of the disease or condition in the subject. As discussed supra, numerous studies have shown aberrant expression of small intestine enzymes such as alkaline phosphatase and lactase in precancerous and cancerous tissues of the esophagus, stomach and large intestine, and other in vitro, non-chemiluminescent (e.g., colorimetric) studies have demonstrated that other enzymes, including glucosidase, glucuronidase, and aryl sulfatase have been characterized in precancerous and cancerous tissues. One need not necessarily refer to studies that compare normal expression of an enzyme with abnormal expression; access to data regarding normal expression of an enzyme may be sufficient to correlate the chemiluminescence to the absence or presence of a disease or condition that is known to be characterized by abnormal expression of the enzyme. Those skilled in the art are familiar with other data that can be used pursuant to correlating chemiluminescence resulting from the reaction of the substrate with the enzyme with the absence or presence of the disease or condition.

The instant methods may also or alternatively comprise correlating chemiluminescence resulting from the reaction of the substrate with the enzyme the stage of disease or condition in the subject, the response of a disease or condition that is known to be present in the subject to a therapy regimen, or both. The presently described methods can yield important information regarding the absence or presence of one or more specific diseases or conditions at a given time or over a period of time, the developmental status of one or more diseases or conditions, the prospective appropriateness of a given therapy regimen, the efficacy of an ongoing therapy regimen, and other matters of importance with regard to the treatment and/or monitoring of a subject. The instant methods may further comprise selecting a therapy regimen based on any of the preceding correlations. The selection of a therapy regimen can embrace initiating a new therapy, altering an ongoing therapy, monitoring of a subject that is or is not undergoing therapeutic intervention, or taking no further action than had been undertaken prior to the comparison. For example, if the correlation reveals that the detected chemiluminescence approaches the upper end of a range of values that would be expected for a subject in which a disease or condition is not present, the selection of a therapy regimen may comprise initiating a protocol whereby the subject is monitored every three months, e.g., by conducting an additional episode of contacting/detecting once every three months and comparing the measured chemiluminescence to a reference value following each clinical visit. In another example, if the correlation reveals that the detected chemiluminescence is greater than what would the minimum amount of chemiluminescence that is expected to indicate the presence of, for example, colon cancer, then such therapeutic intervention as one or more of evaluative colonoscopy, biopsy, blood counts, ultrasound, computed tomography, magnetic resonance imaging, positron emission tomography, angiography, surgery, radiation therapy, chemotherapy, and immunotherapy may be selected pursuant to developing an appropriate therapy regimen. In yet another example, if the detected chemiluminescence reveals that the amount of chemiluminescence is less than a maximum amount of chemiluminescence that would be expected to indicate the presence of, for example, breast cancer, i.e., is less than an amount of chemiluminescence below which there exists an indication of the presence of breast cancer, then appropriate therapeutic intervention such as mammography, biopsy, blood counts, ultrasound, computed tomography, magnetic resonance imaging, positron emission tomography, angiography, surgery, radiation therapy, chemotherapy, and immunotherapy may be selected pursuant to developing an appropriate therapy regimen.

The present methods may also comprise quantifying the chemiluminescence at one or more portions of the tissue. Enzyme expression levels as determined by quantifying the chemiluminescence, i.e., measuring the chemiluminescent intensity, at a portion of the tissue can provide additional information regarding the absence, presence, stage of development, degree, or proliferation of a disease or condition at that portion. The chemiluminescence at a second portion of the tissue may also be quantified. The quantified chemiluminescence at a first portion of the tissue may be compared to the quantified chemiluminescence at a second portion of the tissue. The comparison can provide information regarding the absence, presence, stage of development, degree, or proliferation of a disease or condition at the first portion as compared with the second portion. In addition, enzyme expression pursuant to some diseases and conditions is known to adopt specific patterns within a given tissue; the comparison of the chemiluminescence among various portions of a tissue can be used to identify whether such diseases or conditions are present and in some instances such information as the stage of development, degree, or proliferation of the disease or condition. For example, as demonstrated in Example 1, infra, chemiluminescent activity may be lowest in non-neoplasic tissue, higher in tumor tissue, and higher still in tissue adjacent to tumor tissue. The present methods may further comprise correlating quantified chemiluminescence at one or more tissue portions to the presence or absence of one or more of tumor tissue, tissue adjacent to tumor tissue, and non-tumor tissue.

The quantified chemiluminescence at one or more portions of the tissue may also be compared to a reference value. In accordance with the present invention, the “reference value” may correspond to any of a number of different parameters. For example, the reference value may correspond to the minimum quantity of chemiluminescence that is necessary to indicate the presence of a disease state. The reference value may be a single numerical value expressed in terms of intensity, e.g., relative light units, photon count, etc., with or without a standard deviation, or the reference value may comprise a range of numerical values expressed in terms of, for example, a range of intensity values, with or without standard deviations. The reference value may comprise a single data point, or may comprise an average or mean of multiple data points for the same parameter. For example, the reference value may be the amount of chemiluminescence that was measured in a single sample from a subject known to have a disease or condition, or the reference value may be a calculated average of values obtained from a plurality (e.g., two, five, ten, twenty, one-hundred, etc.) of samples, each from a patient known to have the disease or condition. In other embodiments, the reference value corresponds to the minimum amount of chemiluminescence that is necessary to indicate the presence of the disease or condition, i.e., wherein a sample value that is above such reference value is indicative of the presence of the disease or condition. In such embodiments, the “reference value” is defined as described above. Because it may be difficult, if not medically inappropriate, to attempt to define a single value corresponding to a quantity of chemiluminescence receptor that represents a “tipping point” above or below which (depending on the disease or condition) the disease or condition can definitively be said to be present, a reference value that corresponds to a range of values is preferred. The reference value may also correspond to the amount of chemiluminescence known to be present in the tissue at a known stage of a disease or condition. For example, the reference value may correspond to the amount of chemiluminescence that is known to be present in the tissue at an early stage of colorectal cancer, at a middle stage of colorectal cancer, or at an advanced stage of colorectal cancer.

The “reference value” may also relate to the chemiluminescence at a tissue of the same subject from which the initial detection of chemiluminescence is derived, but at a different point in time. For example, the reference value may correspond to the chemiluminescence at a tissue of a subject at time t=1, whereas the sample value corresponds to the chemiluminescence at a tissue in which a measurement was performed at time t=2, where t=2 represents a later point in time than t=1. The amount of time between times t=1 and t=2 may be one or more hours, days, weeks, months, or years. Additionally, for example, time t=1 may represent a timepoint at which the subject was not undergoing a therapy, whereas time t=2 may represent a timepoint at which a therapy regimen has been commenced.

Because patient prognosis can vary over time, it may be appropriate to perform at least one evaluation of a subject at each of multiple time points. Thus, the present methods can further comprise conducting one or more episodes of contacting tissue of the subject with a substrate for the enzyme and detecting chemiluminescence in the tissue over time. For example, episodes may be performed at timepoint t=0, one or more additional episodes may be performed at timepoint t=1, and a subsequent episode or set of episodes may be performed at timepoint t=2, wherein the amount of time between t=0 and t=1 may be one or more days, weeks, months, or years, and the amount of time between t=1 and t=2 may independently be one or more days, weeks, months, or years. Episodes of “contacting”/“detecting” may be performed a plurality of time points that are equally spaced, or at a plurality of time points that are not equally temporally spaced. Some diseases or conditions may be associated with rapid progression, and based on such factors as the type of disease or condition at issue and/or the patient predisposition for developing the disease or condition, it may be appropriate to perform an episode as often as every few months, every few weeks, or every few days.

A comparison may be made between a reference chemiluminescence value and the initially detected chemiluminescence (which may be referred to as a “sample value”); a comparison may also or alternatively be made between a reference chemiluminescence value and the detected chemiluminescence from one or more of the additional episodes of contacting/detecting (as defined above), between a reference value and an average of detected chemiluminescence values calculated from the detected chemiluminescence from at least two additional episodes of contacting/detecting, or between the sample value and an average of detected chemiluminescence values calculated from the detected chemiluminescence from at least two additional episodes of contacting/detecting. A comparison may also or alternatively be made between the sample value and the detected chemiluminescence from at least one of the additional episodes, or between the detected chemiluminescence from one of the additional episodes to the detected chemiluminescence from another of the additional episodes.

Pursuant to any of such comparisons or any other embodiment disclosed herein, the reference value may correspond to the minimum amount of chemiluminescence that is necessary to indicate the presence of the disease or condition, or the reference value may correspond to the maximum amount of chemiluminescence that is necessary to indicate the presence of the disease or condition, i.e., wherein a measured chemiluminescence value or average of chemiluminescence values that is below such reference value is indicative of the presence of the disease or condition.

A reference value to which the sample chemiluminescence value, the detected chemiluminescence from an additional episode of contacting/detecting, or an average of detected chemiluminescence values calculated from the detected chemiluminescence from at least two additional episodes of contacting/detecting is compared is preferably a “best matched” value, e.g., a sample value that was derived from a given portion of a tissue should be compared with a reference value that was derived from that same portion of tissue, the detected chemiluminescence from an additional episode of contacting/detecting with respect to a particular portion of a tissue should be compared with a sample value and/or reference value that was derived from the same portion of tissue, and the like, depending on the type of comparison being performed.

Pursuant to any of the comparisons described above, the instant methods may further comprise correlating the comparison to the presence or absence of the disease or condition in the subject, the stage of disease or condition in the subject, or the response of a disease or condition that is known to be present in the subject to a therapy regimen. The presently described comparisons can yield important information regarding the absence or presence of one or more specific diseases or conditions at a given time or over a period of time, the developmental status of one or more diseases or conditions, the prospective appropriateness of a given therapy regimen, the efficacy of an ongoing therapy regimen, and other matters of importance with regard to the treatment and/or monitoring of a subject. The instant methods may further comprise selecting a therapy regimen based on any of the preceding comparisons. The selection of a therapy regimen can embrace initiating a new therapy, altering an ongoing therapy, monitoring of a subject that is or is not undergoing therapeutic intervention, or taking no further action than had been undertaken prior to the comparison. For example, if the comparison reveals that the detected chemiluminescence approaches the upper end of a range of values that would be expected for a subject in which a disease or condition is not present, the selection of a therapy regimen may comprise initiating a protocol whereby the subject is monitored every three months, e.g., by conducting an additional episode of contacting/detecting once every three months and comparing the measured chemiluminescence to a reference value following each clinical visit. In another example, if the comparison reveals that the detected chemiluminescence is greater than a reference value that represents the minimum amount of chemiluminescence that is necessary to indicate the presence of, for example, colon cancer, then such therapeutic intervention as one or more of evaluative colonoscopy, biopsy, blood counts, ultrasound, computed tomography, magnetic resonance imaging, positron emission tomography, angiography, surgery, radiation therapy, chemotherapy, and immunotherapy may be selected pursuant to developing an appropriate therapy regimen. In yet another example, if the comparison between the detected chemiluminescence and the reference value reveals that the amount of chemiluminescence is less than a reference value that represents a maximum amount of chemiluminescence that is necessary to indicate the presence of, for example, breast cancer, i.e., is less than an amount of chemiluminescence below which there exists an indication of the presence of breast cancer, then appropriate therapeutic intervention such as mammography, biopsy, blood counts, ultrasound, computed tomography, magnetic resonance imaging, positron emission tomography, angiography, surgery, radiation therapy, chemotherapy, and immunotherapy may be selected pursuant to developing an appropriate therapy regimen.

### EXAMPLES

### Example 1

### Chemiluminescence-Enhanced Endoscopy For Detection of Cancerous and Precancerous Tissue

Methods. Human tissues were harvested under an institutional review board (IRB) approved protocol and had been snap-frozen and stored at −80° C. Tissue samples were thawed at room temperature and processed to provide tissue samples of approximately 0.1×0.1×0.1 cm in size. Tissues were deposited in triplicate into a multiwell assay plate.

Chemiluminescent substrates solutions were prepared as a mixture of substrate (either CSPD or Galacton-Plus; Tropix, MA), and chemiluminescent enhancer (either SapphireI or SapphireII; Tropix, MA) suspended in a Tris-HCl buffered solution.

Chemiluminescent substrates solutions were prepared fresh daily and 100 μA of solution was added to each sample well containing tissue. In addition to the tissue samples, a triplicate of blank wells were run for normalization (blank wells contained the substrate solution with no tissue). The samples were incubated with the substrate solutions and then the multiwell plate was measured for luminescence on a Berthold microplate luminometer LB 960 (Berthold; Wild Bad, Germany). Raw luminescence data was averaged from the triplicate assays and then normalized into relative light units (average of triplicate assays divided by the average of a triplicate blank control).

The relative light unit data for the tumors and tissue adjacent to tumor were subjected to statistical analysis, specifically, ANOVA and student t-test.

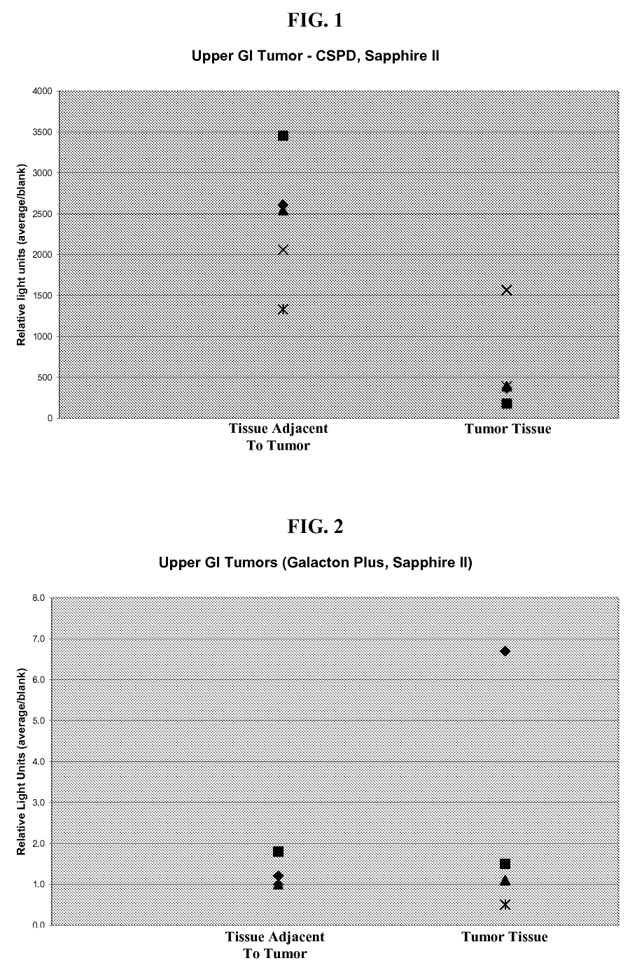

Results. Three samples of esophageal adenocarcinoma, two samples of gastric adenocarcinoma, five samples of tissues adjacent to esophageal and gastric adenocarcinoma, and five gastric samples from gastric reduction or pancreatic cancer resection were assayed with CSPD/SapphireII (Table 1, FIG. 1) or Galacton-Plus/SapphireII (Table 2, FIG. 2).

Table 1, below, provides data for CSPD/SapphireII chemiluminescence in upper gastrointestinal tract tumors, and non-esophageal or gastric tumor resection tissue. All values are expressed as relative light units.

TABLE 1

| | Tissue adjacent | | Non-GI |
|---|---|---|---|
| | to tumor | Tumor | tumor resection |
| Esophageal | 2605.5 | 365.1 | |
| adenocarcinoma | | | |
| Esophageal | 3455.1 | 176.8 | |
| adenocarcinoma | | | |
| Gastric | 2544.9 | 389.6 | |
| adenocarcinoma | | | |
| Gastric | 2059.2 | 1566.4 | |
| adenocarcinoma | | | |
| Esophageal | 1330.7 | 386.5 | |
| adenocarcinoma | | | |
| Stomach with | | | 449.3 |
| chronic gastritis | | | |
| Stomach from | | | 2848.1 |
| pancreatic resection | | | |
| Stomach from | | | 2910.7 |
| pancreatic resection | | | |
| Stomach with | | | 338.2 |
| intestinal metaplasia | | | |
| and chronic gastritis | | | |
| Stomach from | | | 1677.7 |
| pancreatic resection | | | |

Table 2, below, provides data for Galacton-Plus/SapphireII chemiluminescence in upper gastrointestinal tract tumors and adjacent tissue. All values are expressed as relative light units.

TABLE 2

| | Tissue adjacent | | |
|---|---|---|---|
| | to tumor | Tumor | Non-tumor |
| Esophageal | 1.2 | 6.7 | |
| adenocarcinoma | | | |
| Esophageal | 1.8 | 1.5 | |
| adenocarcinoma | | | |
| Gastric | 1.0 | 1.1 | |
| adenocarcinoma | | | |
| Gastric | 1.1 | 0.5 | |
| adenocarcinoma | | | |
| Esophageal | 1.1 | 0.5 | |
| adenocarcinoma | | | |
| Stomach with | | | 0.4 |
| chronic gastritis | | | |
| Stomach from | | | 1.2 |
| pancreatic resection | | | |
| Stomach from | | | 0.9 |
| pancreatic resection | | | |
| Stomach with | | | 0.9 |
| intestinal metaplasia | | | |
| and chronic gastritis | | | |
| Stomach from | | | 3.9 |
| pancreatic resection | | | |

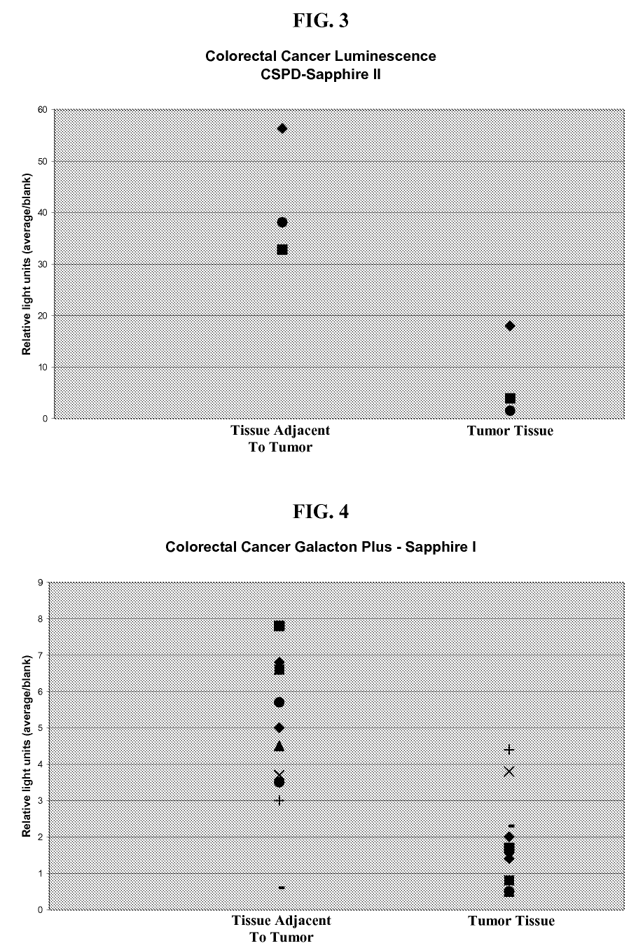

Three samples of colorectal adenocarcinoma, and three samples of tissues adjacent to colorectal adenocarcinoma were assayed with CSPD/SapphireII (Table 3, FIG. 3). Ten samples of colorectal adenocarcinoma, ten samples of tissues adjacent to colorectal adenocarcinoma, and two samples of colon from non-tumor resections were assayed with Galacton-Plus/SapphireI (Table 4, FIG. 4).

Table 3, below, provides data for CSPD/SapphireII chemiluminescence in colorectal adenocarcinomas and adjacent tissue. All values are expressed as relative light units.

TABLE 3

| | Tissue adjacent | Colorectal |
|---|---|---|
| | to tumor | adenocarcinoma |
| 22660 | 56.3 | 18 |
| 18878 | 32.8 | 3.9 |
| 14457 | 38.1 | 1.5 |

Table 4, below, provides data for Galacton-Plus/SapphireI chemiluminescence in colorectal adenocarcinomas and adjacent tissue. All values are expressed as relative light units.

TABLE 4

| | Tissue Adjacent | | Non-tumor |
|---|---|---|---|
| | to Tumor | Tumor | resection |
| Colorectal adenocarcinoma | 5 | 2 | |
| Colorectal adenocarcinoma | 7.8 | 1.7 | |
| Colorectal adenocarcinoma | 4.5 | 0.5 | |
| Colorectal adenocarcinoma | 3.7 | 3.8 | |
| Colorectal adenocarcinoma | 6.6 | 0.8 | |
| Colorectal adenocarcinoma | 3.5 | 0.5 | |
| Colorectal adenocarcinoma | 3.0 | 4.4 | |
| Colorectal adenocarcinoma | 0.6 | 2.3 | |
| Colorectal adenocarcinoma | 5.7 | 1.6 | |
| Colorectal adenocarcinoma | 6.8 | 1.4 | |
| Prolapsed colon | | | 0.7 |
| Diverticulosis | | | 1.8 |

Interpretation of Upper Gastrointestinal Assays. Chemiluminescence activity for alkaline phosphatase (CSPD/SapphireII) in the upper gastrointestinal tissues adjacent to tumor was higher than both the tumors as well as upper gastrointestinal tissues not associated with esophageal or gastric tumors (p=0.025; ANOVA analysis; http://www.physics.csbsju.edu/cgi-bin/stats/anova). The alkaline phosphatase activity of both tumors and tissue adjacent to tumors appear to be somewhat higher than gastric tissues from gastric reduction and pancreatic cancer resections.

Similar ANOVA analysis for chemiluminescence activity for lactase/Beta-galactosidase (Galacton-Plus/SapphireII) revealed no statistical difference (p=0.745); however, there was one example of an esophageal tumor that was five-fold higher than its matched adjacent tissue.

Thus, for upper gastrointestinal tissues (esophagus and stomach) it appears that tissue adjacent to tumors and tumors themselves both have chemiluminescent activity detectable by substrates for alkaline phosphatase and lactase/beta-galactosidase. Upper gastrointestinal tissues adjacent to tumors have statistically higher alkaline phosphatase activity compared to tumors. This activity may also be higher than non-GI tumor related gastric resection tissue. This activity pattern may be useful in that true non-neoplastic upper gastrointestinal tissues may have the lowest activity, followed by frank carcinoma, and then with the highest activity by tissue adjacent to the tumor. This pattern may present as a “bulls-eye” with an increased chemiluminescent activity at the rim of the tumor and the actual tumor proper with a decreased chemiluminescent activity.

Interpretation of Colorectal Assays. Chemiluminescence activity for alkaline phosphatase (CSPD/SapphireII) in the colorectal tissues adjacent to tumor was higher than the tumors (p=0.017; student t-test analysis).

Similarly, chemiluminescence activity for lactase/beta-galactosidase (Galacton-Plus/Sapphire I) in the colorectal tissues adjacent to tumor was higher than the tumors (p=0.002; student-test analysis). The chemiluminescence activity in the two non-tumor diverticulosis and prolapase specimens appear to be generally lowest activity.

Thus, for colorectal tissues it appears that tissue adjacent to tumors has a statistically higher alkaline phosphatase and lactase/beta-galactosidase activity compared to tumors. This activity may also be higher than that found in non-neoplastic colons from non-tumor resections (prolapse, diverticulosis). This activity may be useful in that true non-neoplastic colorectal tissues may have the lowest activity, followed by frank carcinoma, and then with the highest activity in tissues adjacent to tumor. This pattern is similar to that demonstrated in upper gastrointestinal tissue and appears to be a “bull's-eye” with an increased chemiluminescent activity at the rim of the tumor and the actual tumor proper with a decreased chemiluminescent activity.

This type of “bull's eye” pattern was originally described in gastric cancers that were examined for sucrase enzyme activity (Kawachi T, et al., Cancer Res. 1976 July; 36(7 PT 2):2673-7). In this prior study, chromogenic precipitates were produced as a result of sucrase activity in gastric tissues adjacent to tumor. In this colorimetric assay, tumors were presumed to be negative for enzyme activity. The high activity surrounding the tumor was presumed to be secondary to intestinal metaplasia. The present study demonstrates that the tumors have a reduced but maintained level of enzymatic activity as detected by the more sensitive chemiluminescent method. The present studies further demonstrate, inter alia, that the chemiluminescent substrates for alkaline phosphatase and lactase/beta-galactosidase are sensitive detection reagents for the detection of precancerous and cancerous lesions of the gastrointestinal tract.

What is claimed:

1. A method for assessing a disease or a condition in a subject, which disease or condition is known to be characterized by abnormal expression of an enzyme, said method comprising:
contacting tissue of said subject with a substrate for said enzyme, said substrate chemiluminescing upon reaction with said enzyme;
detecting chemiluminescence in said tissue; and,
correlating said chemiluminescence to at least one of the presence or absence of said disease or condition in said subject, the stage of said disease or condition in said subject, or the response of said disease or condition in said subject to a therapy regimen.

2. The method according to claim 1 wherein said substrate produces chemiluminescence upon reaction with one or more of alkaline phosphatase, lactase (beta-galactosidase), glucosidase, glucoronidase, aryl sulfatase, trehalase, maltase, dipeptidyl peptidase-IV, aminopeptidase N, endopeptidase F, and sucrase-isomaltase.

3. The method according to claim 1 wherein said substrate comprises an adamantyl dioxetane.

4. The method according to claim 3 wherein said substrate comprises an adamantyl 1,2-dioxetane aryl phosphate.

5. The method according to claim 1 wherein said abnormal expression comprises elevated expression as compared with a wild type level of expression.

6. The method according to claim 1 wherein said abnormal expression comprises decreased expression as compared with a wild type level of expression.

7. The method according to claim 1 wherein said disease or condition comprises neoplasia.

8. The method according to claim 1 wherein said disease or condition comprises precancer or cancer.

9. The method according to claim 1 wherein said tissue comprises esophageal, gastric, colorectal, or intestinal tissue.

10. The method according to claim 1 wherein said tissue is in the oral cavity, sinonasal cavity, oropharynx, respiratory tract, liver, bile ducts, gall bladder, pancreatic ducts, urethra, bladder, ureter, renal pelvis, vagina, cervix, uterus, peritoneal cavity, or pleural cavity.

11. The method according to claim 1 wherein said contacting comprises administering a dose of said substrate to said subject.

12. The method according to claim 1 further comprising contacting said tissue with an enhancer molecule.

13. The method according to claim 1 further comprising contacting said tissue with an additional substrate for said enzyme.

14. The method according to claim 1 further comprising contacting said tissue with a substrate for a second enzyme.

15. The method according to claim 1 further comprising contacting said tissue with a contrast agent.

16. The method according to claim 1 wherein said chemiluminescence is detected using an endoscope.

17. The method according to claim 16 wherein said endoscope is configured for effecting confocal microscopy using said endoscope.

18. The method according to claim 1 further comprising selecting a therapy regimen based on any of said correlations.

19. The method according to claim 1 wherein the chemiluminescence is quantified at one or more portions of said tissue.

20. The method according to claim 19 further comprising comparing the quantified chemiluminescence of a first portion of said tissue to the quantified chemiluminescence of a second portion of said tissue.

21. The method according to claim 19 further comprising correlating the quantified chemiluminescence at said one or more tissue portions to the presence or absence of one or more of tumor tissue, tissue adjacent to tumor tissue, and non-tumor tissue.

22. The method according to claim 19 further comprising comparing the quantified chemiluminescence at one or more portions of said tissue to a reference value.

23. The method according to claim 22 wherein said reference value corresponds to the minimum amount of chemiluminescence that is necessary to indicate the presence of said disease or condition.

24. The method according to claim 22 wherein said reference value corresponds to the amount of chemiluminescence known to be present in said tissue at a known stage of said disease or condition.

25. The method according to claim 22 further comprising correlating said comparison to the presence or absence of said disease or condition in said subject, the stage of said disease or condition in said subject, or the response of said disease or condition in said subject to a therapy regimen.

26. The method of claim 1 further comprising conducting one or more additional episodes of said contacting and detecting steps over time.

27. The method according to claim 26 further comprising comparing the initially detected chemiluminescence to the detected chemiluminescence from at least one of said additional episodes, comparing the detected chemiluminescence from one of said additional episodes to the detected chemiluminescence from another of said additional episodes, comparing the initially detected chemiluminescence to an average chemiluminescence value that is calculated from the chemiluminescence from two or more additional episodes, comparing an average chemiluminescence value that is calculated from the chemiluminescence from two or more additional episodes to a reference value, or any combination thereof.

28. The method according to claim 27 comprising correlating any of said comparisons to the presence or absence of said disease or condition in said subject, the stage of said disease or condition in said subject, or the response of said disease or condition in said subject to a therapy regimen.

29. The method according to claim 28 further comprising selecting a therapy regimen based on any of said correlations.
